(19) United States
(12) Patent Application Publication
(10) Pub. No.: US 2024/0408289 A1
(43) Pub. Date: Dec. 12, 2024

(54) APPARATUS FOR EXTRACORPOREAL BLOOD TREATMENT

(71) Applicant: GAMBRO LUNDIA AB, Lund (SE)

(72) Inventor: Innas Forsal, Malmö (SE)

(21) Appl. No.: 18/701,681

(22) PCT Filed: Oct. 11, 2022
(86) PCT No.: PCT/EP2022/078290

§ 371 Date: Apr. 16, 2024

(30) Foreign Application Priority Data
Nov. 4, 2021 (IT) 102021000028082

(51) Int. Cl. A61M 1/16; A61M 1/36

(52) U.S. Cl. CPC A61M 1/1611; A61M 1/1631; A61M 1/367; A61M 2202/0007; A61M 2202/0021; A61M 2202/0413; A61M 2202/0486; A61M 2205/10; A61M 2205/3303; A61M 2205/3327; A61M 2205/3334; A61M 2205/50; A61M 2230/005; A61M 2230/04; A61M 2230/207

(57) ABSTRACT

An apparatus for extracorporeal blood treatment comprises a control unit (100) connected to a blood pump (10) configured to deliver a blood flow rate in a blood circuit of the apparatus (1), to a diuretic pump (27) configured to deliver a flow rate (Q_{d}) of a 5diuretic (e.g. furosemide) and to an osmotic agent pump (30) configured to deliver a flow rate (Qₒₐ) of an osmotic agent (e.g. albumin) to be infused in the blood circuit or in the vascular system of the patient (P). The control unit (100) is configured for receiving at least one input patient parameter (e.g. blood 10pressure) and/or at least one input apparatus parameter (e.g. access pressure) and, during an extracorporeal blood treatment, to drive the diuretic pump (27) and/or the osmotic agent pump (30) as a function of said at least one input patient parameter and/or as a function of at least one input apparatus parameter, in order to15achieve an improved and better fluid removal from the patient (P).

### TECHNICAL FIELD

The present invention relates to an apparatus for extracorporeal blood treatment. The present disclosure also relates to a method for controlling a patient fluid removal in an apparatus for extracorporeal blood treatment.

Extracorporeal blood treatment involves removing blood from a patient, treating the blood externally to the patient, and returning the treated blood to the patient. Extracorporeal blood treatment is typically used to extract undesirable matter or molecules from the patient's blood and add desirable matter or molecules to the blood. Extracorporeal blood treatment is used with patients unable to effectively remove matter from their blood, such as when a patient has suffered temporary or permanent kidney failure. These patients and other patients may undergo extracorporeal blood treatment to add or remove matter to their blood, to maintain an acid/base balance, to remove excess body fluids or to perform extracorporeal gas exchange processes, for example.

The present invention refers to the treatment of fluid overload of patients through extracorporeal blood treatments, in particular but not exclusively in continuous renal replacement therapy (CRRT) systems. CRRT systems are configured for delivering treatments designed for patients versing in acute states of illness and who have temporarily lost their kidney function in its entirety. CRRT monitors should be able to deliver various therapies, e.g.: ultrafiltration (UF), slow continuous ultrafiltration (SCUF), continuos veno venous hemofiltration (CCVH), continuos veno venous hemodiafiltration (CVVHDF).

The invention may also be applied to chronic (long-term) hemodialysis (HD) therapy systems. Chronic (long-term) hemodialysis (HD) therapy systems are used to treat patients with chronic kidney failure. HD therapy is usually performed several times a week (e.g. 3 times a week) and each treatment has an average duration of some hours (e.g. 3.5-4.5 hours).

Notably, the present invention may also be used in connection with Congestive Heart Failure (CHF) treatments; congestive heart failure (CHF) is a set of manifestations caused by the failure of the heart's function as a pump supporting the blood flow through the body. Filling dysfunction and high intracardiac pressure may result in the buildup of fluid in the veins and tissues. This manifests as water retention and swelling due to the buildup of liquid (edema), collectively referred to as congestion.

Ultrafiltration treatments are used to remove excess water, particularly in case diuretics have no or little effects or when the patient has no or residual renal function.

### Background of the Invention

Conventional treatment of fluid overload is the usage of diuretic and measuring the patient's weight until a goal weight is achieved.

When a diuretic is administered, fluid is recruited directly from the vascular volume via the kidney and if this rate exceeds the rate that the resultant oncotic pressure can recruit fluid from the interstitial space (splanchnic and somatic volumes), the volume in the vascular space is depleted. This replenishment rate is known as the PRR (Plasma Refill Rate).

The problem with diuretics is that, if the patient has no urine production, no fluid will be removed. Furthermore, if the fluid is located peripherally in the interstitial space, the increase of diuretics does not improve excess fluid removal but may cause hypotension to the patient and/or renal failure.

Continuous renal replacement therapy (CRRT) may also be used for treatment of fluid overload. These are quite long treatments with slow but prolonged removal of liquids.

Unfortunately, also CRRT presents the same problem related to the peripherally located fluid. There have been tricks that Medical Doctors have done to get better removal of excess fluids through CRRT and two of these is usage of albumin as an osmotic agent to force fluid into the central circulation and diuretics infusion or oral administration of diuretics to remove fluid through urine.

Document US 2007/0055198 is also known, which discloses a blood volume control method comprising: monitoring a condition of a patient indicative of blood volume in the patient, determining that the condition indicates a low blood volume in the patient; infusing an infusion solution into the patient in response to the determination of a low blood volume and adjusting the infusion to maintain the monitored condition at a predetermined value. The method disclosed in US 2007/0055198 also provides for infusing furosemide in the patients' peripheral blood vessels in the forearm or centrally in the venous system and for increasing artificially the Plasma Refill Rate (PRR) by the infusion of a Hypertonic Saline Solution or other such blood volume expander such as a dextrose solution, sodium bicarbonate solution or albumin using a standard syringe and IV needle.

There is a desire to further improve known extracorporeal blood treatments for the removal of excess fluid using diuretics and/or osmotic agents.

An aim of the present invention is therefore to provide for an apparatus for extracorporeal blood treatment and a method for controlling a patient fluid removal that allow to achieve a better fluid removal and a better treatment to the patient.

It is an aim of the present invention to provide for an apparatus for extracorporeal blood treatment and a method for controlling a patient fluid removal that allow to reduce risks of criticalities in patients undergoing treatment.

It is an aim of the present invention to provide a method for controlling a patient fluid removal which is automatically executed during an extracorporeal blood treatment.

It is an aim of the present invention to tailor the treatment and in particular the treatment of fluid overload to each specific patient and to each specific extracorporeal blood treatment.

It is a further aim of the present invention to improve the comfort of patients undergoing treatment.

It is a further aim of the present invention to reduce the workload of the clinical staff and/or of the Medical Doctors due in particular to problems linked to fluid removal.

### SUMMARY

An apparatus according to one or more of the appended claims, taken singly or in any combination, attains at least one of the above-indicated aims.

The applicant noted that known extracorporeal blood treatments for the removal of excess fluid using diuretics and/or osmotic agents may be improved by tailoring the infusion of said diuretics and/or osmotic agents to the specific patient and type of extracorporeal blood treatment in order to achieve an improved fluid removal.

The present invention provides an extracorporeal blood treatment apparatus and a method for controlling a patient fluid removal which are able to automatically command the apparatus and control an infusion of a diuretic and/or of an osmotic agent as a function of one or more input patient parameters and/or one or more apparatus parameters.

An apparatus and a method according to aspects of the invention and capable of achieving one or more of the above aims are here below described.

In a 1st independent aspect there is provided an apparatus for extracorporeal blood treatment, comprising:
a treatment unit;
a blood circuit coupled to the treatment unit and comprising a blood removal line and a blood return line connectable to a vascular system of a patient;
a blood pump configured to be coupled to a pump section of the blood circuit to deliver a blood flow rate;
an effluent line connected to the treatment unit;
an effluent pump configured to be coupled to a pump section of the effluent line to deliver an effluent flow rate;
a source of at least one diuretic coupled to a diuretic pump and connected to the blood circuit or connectable to the vascular system of the patient, wherein the diuretic pump is configured to deliver a diuretic flow rate;
a source of at least one osmotic agent coupled to an osmotic agent pump and connected to the blood circuit or connectable to the vascular system of the patient, wherein the osmotic agent pump is configured to deliver an osmotic agent flow rate;
a control unit connected at least to the blood pump, the diuretic pump and the osmotic agent pump and configured and/or programmed for receiving at least one input patient parameter and/or at least one input apparatus parameter;
wherein, during an extracorporeal blood treatment, the control unit is programmed for driving at least the diuretic pump and/or the osmotic agent pump as a function of said at least one input patient parameter and/or as a function of at least one input apparatus parameter.

The control unit is configured and/or programmed for implementing a script or algorithm for controlling a patient fluid removal in the apparatus for extracorporeal blood treatment.

In a 2nd independent aspect there is provided a method for controlling a patient fluid removal in an apparatus for extracorporeal blood treatment, comprising:
setting a prescription value and/or detecting measured values of at least one input patient parameter and/or at least one input apparatus parameter of an apparatus for extracorporeal blood treatment;
during an extracorporeal blood treatment, driving at least a diuretic pump and/or an osmotic agent pump of the apparatus for extracorporeal blood treatment or connected to the apparatus for extracorporeal blood treatment as a function of said at least one input patient parameter and/or as a function of at least one input apparatus parameter.

In a 3rd aspect, the apparatus of the 1^{st} aspect or of any of the following aspects is configured to perform a method for controlling a patient fluid removal, optionally the method of the 2^{nd} aspect or of any of the following aspects, or the method of the 2^{nd} aspect or of any of the following aspects is implemented through the apparatus of the 1^{st} aspect or of any of the following aspects.

In a 4^{th} aspect according to one or more of the previous aspects, the at least one input patient parameter comprises: heart rate and/or blood pressure and/or urine output and/or consciousness level and/or body weight and/or creatinine and/or hematocrit and/or level of electrolytes; optionally, one or more of said input patient parameters are laboratory values (e.g. creatinine or body weight or blood pressure); optionally one or more of said input patient parameters are measured during treatment (e.g. urine output and/or consciousness level and/or heart rate and/or hematocrit).

In a 5^{th} aspect according to one or more of the previous aspects, the at least one input apparatus parameter comprises: blood flow rate and/or effluent flow rate and/or filtration flow rate and/or access pressure and/or return pressure and/or fluid removal goal and/or total patient weight loss and/or total treatment time.

In a 6^{th} aspect according to one or more of the previous aspects, the apparatus for extracorporeal blood treatment is a continuous renal replacement therapy (CRRT) system for intensive care treatments.

In a 7^{th} aspect according to one or more of the previous aspects, the apparatus for extracorporeal blood treatment further comprises:
at least one infusion line connected to the blood circuit and to a source of at least one infusion fluid;
an infusion pump configured to be coupled to a pump section of said at least one infusion line and to deliver an infusion flow rate.

In a 8^{th} aspect according to the previous aspect, the at least one input apparatus parameter further comprises the infusion flow rate.

In a 8^{th} bis aspect according to one or more of the previous aspects, the apparatus for extracorporeal blood treatment further comprises:
at least one auxiliary line connected to the blood circuit and to a source of at least one compensation substance, such as potassium or bicarbonate;
a pump or syringe configured to be coupled to a pump section of said at least one auxiliary line and to deliver a flow rate of the compensation substance.

In a 8^{th} ter aspect according to the previous aspect, the at least one input apparatus parameter further comprises the flow rate of the compensation substance.

In a 9^{th} aspect according to one or more of the previous aspects, the apparatus for extracorporeal blood treatment further comprises:
a dialysis line connected to the treatment unit and to a source of a dialysis fluid;
a dialysis pump configured to be coupled to a pump section of the dialysis line and to deliver a dialysis flow rate.

In a 10^{th} aspect according to the previous aspect, the at least one input apparatus parameter further comprises the dialysis flow rate.

In a 11^{th} aspect according one or more of the previous aspects, the apparatus comprises or is connected to at least one sensor connected to the control unit, the at least one sensor being configured to detect measured values of the at least one input patient parameter and/or the at least one input apparatus parameter; optionally the control unit and the at least one sensor are configured to monitor continuously the at least one input patient parameter and/or the at least one input apparatus parameter.

In a 12^{th} aspect according to the previous aspect, the at least one sensor is configured to detect the measured values of at least one of the following input patient parameters: heart rate, blood pressure, urine output, consciousness level, body weight, creatinine, hematocrit, level of electrolytes.

In a 13^{th} aspect according to the previous aspect 11 or 12, the at least one sensor is configured to detect the measured values of at least one of the following input apparatus parameters: blood flow rate, effluent flow rate, filtration flow rate, access pressure, return pressure, difference between the access pressure and the return pressure, dialysis flow rate, infusion flow rate.

In a 14^{th} aspect according one or more of the previous aspects, the control unit is configured and/or programmed for receiving a prescription value of the at least one input patient parameter and/or the at least one input apparatus parameter.

In a 15^{th} aspect according to the previous aspect, the control unit is configured and/or programmed for receiving the prescription value of the at least one of the following input apparatus parameter: blood flow rate, effluent flow rate, filtration flow rate, dialysis flow rate, infusion flow rate.

In a 15^{th} bis aspect, according to the previous aspect 14 or 15, the control unit is configured and/or programmed for receiving the total patient weight loss and/or the fluid removal goal and/or the total treatment time.

In a 16^{th} aspect according to one or more of the previous aspects 11 to 15, the control unit is configured and/or programmed for, or the method comprises: monitoring said measured values over time and driving at least the diuretic pump and/or the osmotic agent pump as a function of variation over time of said measured values.

In a 17^{th} aspect according to one or more of the previous aspects 11 to 15, the control unit is configured and/or programmed for, or the method comprises: driving at least the diuretic pump and/or the osmotic agent pump as a function of the prescription value of the input patient parameter and/or the input apparatus parameter, optionally as a function of a comparison between said prescription value with the measured values of the input patient parameter and/or the input apparatus parameter.

In an 18^{th} aspect, according to one or more of the previous aspects, it is provided for pausing antihypertensive medication during the extracorporeal blood treatment.

In a 19^{th} aspect according to aspect 5 or 13, the control unit is configured and/or programmed for performing the following procedure or the method comprises:
if the access pressure is above a high access pressure threshold, for instance of 250 mmHg or of 450 mmHg, or within an access pressure range, for instance between 100 mmHg and 450 mmHg, optionally between 160 mmHg and 250 mmHg, (i.e. an access pressure range not triggering alarm for access pressure) commanding the effluent pump to increase the effluent flow rate and the filtration flow rate;
if the access pressure is below a low access pressure threshold (for instance of 100 mmHg or 160 mmHg), commanding the blood pump to reduce a blood flow rate and/or commanding the effluent pump to reduce the effluent flow rate and the filtration flow rate and/or commanding the osmotic agent pump and/or the diuretic pump to reduce or increase the diuretic flow rate and/or the osmotic agent flow rate.

In a 20^{th} aspect according to aspect 4 or 12 and according to aspect 5 or 13, the control unit is configured and/or programmed for performing the following procedure or the method comprises:
if, at the start of the extracorporeal blood treatment (at instant t=0) the access pressure is above a high access pressure threshold or within an access pressure range and the blood pressure is above a blood pressure threshold (e.g. set by physician, e.g. greater than 100 mmHg systolic or e.g. comprised between80 mmHg and 120 mmHg), then commanding the blood pump to increase the blood flow rate and/or commanding the effluent pump to increase the effluent flow rate and the filtration flow rate and/or commanding the diuretic pump to start or increase the diuretic flow rate (e.g. furosemide infusion);
if, at the start of the extracorporeal blood treatment (at instant t=0) the access pressure is below a low access pressure threshold and the blood pressure is below a blood pressure threshold (e.g. below 100 mmHg systolic), then commanding the blood pump to decrease the blood flow rate and/or commanding the effluent pump to decrease the effluent flow rate and the filtration flow rate and/or commanding the osmotic agent pump to start or increase the osmotic agent (e.g. giving an albumin bolus) and/or commanding the diuretic pump to start or increase the diuretic flow rate (e.g. solution iv).

In a 21^{st} aspect according to any of aspects 5, 13, 19 or 20, the control unit is configured and/or programmed for or the method comprises: commanding the apparatus to send an alarm signal if the access pressure is below a low alarm access pressure threshold or above a high alarm access pressure threshold.

In a 22ⁿd aspect according to aspect 4 or 12 and according to any of aspects 5, 13, 19, 20 or 21, the control unit is configured and/or programmed for performing the following procedure or the method comprises:
i. if the access pressure decreases over time (i.e. if the access pressure at instant t=0 is greater than the access pressure at instant t=1), then starting the diuretic pump and/or the osmotic agent pump to deliver the diuretic flow rate and/or the osmotic agent flow rate (i.e. to give diuretic and/or osmotic agent to the patient);
ii. if the access pressure increases over time (e.g. if the access pressure at instant t=1 is greater than the access pressure at instant t=0), taking no action or commanding the effluent pump to increase the effluent flow rate and the filtration flow rate, in order to increase fluid removal from patient.

In a 23^{rd} aspect according to the previous aspect 22, if situation i. occurs, the control unit is configured and/or programmed for performing the following procedure or the method comprises:
if the access pressure keeps on decreasing over time (e.g. the access pressure at instant t=1 is greater than the access pressure at instant t=2), then starting the osmotic agent pump and/or the diuretic pump or commanding the diuretic pump to increase the diuretic flow rate and/or commanding the osmotic agent pump to increase the osmotic agent flow rate; or
if the access pressure starts increasing over time (e.g. if the access pressure at instant t=2 is greater than the access pressure at instant t=1), then commanding the diuretic pump to maintain the diuretic flow rate and/or commanding the osmotic agent pump to maintain the osmotic agent flow rate.

In a 24^{th} aspect according to aspect 4 or 12, if the blood pressure is within a blood pressure range, the control unit is configured and/or programmed for or the method comprises: commanding the blood pump to increase the blood flow rate, commanding the diuretic pump to increase the diuretic flow rate and commanding the effluent pump to increase the effluent flow rate and the filtration flow rate.

In a 25^{th} aspect according to aspect 4 or 12, if the blood pressure is below a blood pressure threshold, the control unit is configured and/or programmed for or the method comprises: starting the osmotic agent pump or commanding the osmotic agent pump to increase the osmotic agent flow rate till the blood pressure is stabilized and then starting the diuretic pump or commanding the diuretic pump to increase the diuretic flow rate.

In a 26^{th} aspect according to aspect 4 or 12 or 24 or 25 and to one or more of aspects 5, 13, 19-23, if at the start of the extracorporeal blood treatment the creatinine is above a creatinine threshold indicating Chronic Kidney Disease (CKD), the control unit is configured and/or programmed for or the method comprises: commanding the diuretic pump to increase the diuretic flow rate.

In a 27^{th} aspect according to aspect 26, the control unit is configured and/or programmed for or the method comprises:
if the creatinine starts decreasing over time and/or the urine output starts decreasing over time, then commanding the diuretic pump to increase the diuretic flow rate and/or commanding the osmotic pump to start;
if the creatinine starts increasing over time and/or the urine output starts decreasing over time, then commanding the diuretic pump to decrease the diuretic flow rate and/or commanding the infusion pump to start the infusion flow rate.

In a 28^{th} aspect according aspect 27, if the creatinine starts increasing over time and/or the urine output starts decreasing over time and hematocrit increases, then add glucose or a buffer solution or any solution prescribed, such as saline.

In a 29^{th} aspect according to any of aspects 4, 12 or 24 to 28 and to one or more of aspects 5, 13, 19-23, if one or more of the following critical conditions occur:
creatinine is outside a creatinine range;
hematocrit is outside a hematocrit range;
then the control unit is configured and/or programmed for or the method comprises:
performing a less aggressive treatment, e.g. commanding the blood pump to reduce a blood flow rate and/or commanding the effluent pump to reduce the effluent flow rate and the filtration flow rate and/or commanding the osmotic agent pump and/or the diuretic pump to reduce or increase the diuretic flow rate and/or the osmotic agent flow rate.

In a 30^{th} aspect according to any of aspects 4, 12 or 24 to 29 and to one or more of aspects 5, 13, 19-23, the control unit is configured and/or programmed for performing the following procedure or the method comprises:
if hematocrit increases over time (e.g. if hematocrit at instant t=0 is lower than hematocrit at instant t=1), then commanding the diuretic pump to decrease the diuretic flow rate; or
if the urine output increases over time (e.g. if urine output at instant t=0 is lower than urine output at instant t=1), then commanding the infusion pump to start or to increase the infusion flow rate; and then
if the urine output still increases over time and hematocrit starts decreasing, then maintain the infusion flow rate.

In a 31^{st} aspect according to any of aspects 4, 12 or 24 to 30 and to one or more of aspects 5, 13, 19-23, the method comprises: if the access pressure is above a high access pressure threshold, the hematocrit is above a high hematocrit threshold and the return access pressure is below a low return pressure threshold, then check access for possible clot formation.

In a 32^{nd} aspect according to the previous aspects 4, 5 and 7 or 8, if the blood pressure is below the blood pressure threshold and access pressure is outside an access pressure range, the control unit is configured and/or programmed for or the method comprises: commanding the infusion pump to start infusion flow rate, optionally of saline or glucose.

In a 33^{rd} aspect according to aspect 4 or 12, if the urine output is below an urine threshold and the blood pressure is within the blood pressure range, the control unit is configured and/or programmed for or the method comprises: commanding the effluent pump to increase the effluent flow rate and the filtration flow rate and commanding the diuretic pump to maintain the diuretic flow rate.

In a 34^{th} aspect to one or more of the previous aspects, the control unit is configured and/or programmed for receiving data of the diuretic (e.g. type of diuretic used) and/or data of the osmotic agent (e.g. type of osmotic agent used).

In a 35^{th} aspect to one or more of the previous aspects, the osmotic agent comprises albumin or amino acids or mannitol or hypertonic saline or glucose solution.

In a 36^{th} aspect to one or more of the previous aspects, the diuretic comprises furosemide or torsemide or bumetanide or ethacrynic acid or metolazone or chlorthalidone or hydrochlorothiazide or chlorothiazide or spironolactone or eplererone; optionally the diuretic is a solution of sodium chloride mixed with furosemide.

In a 37^{th} aspect to one or more of the previous aspects, the apparatus for extracorporeal blood treatment comprises a treatment machine, wherein the treatment machine comprises the control unit and the blood pump; optionally the treatment machine comprises the dialysis pump and the infusion pump/s.

In a 38^{th} aspect to the previous aspect, the treatment machine comprises the osmotic agent pump and/or the diuretic pump.

In a 39^{th} aspect according to aspect 37, the osmotic agent pump and/or the diuretic pump is/are external to the treatment machine; optionally the osmotic agent pump and/or the diuretic pump is/are part of independent device/s and not part of the treatment machine.

In a 40^{th} aspect according to aspect 37, 38 or 39 and aspects 7 to 10, the apparatus for extracorporeal blood treatment comprises an integrated disposable set, wherein the treatment unit, the blood circuit, the effluent line, optionally also the dialysis line and the infusion line/s are grouped together and connected in the integrated disposable set.

In a 41^{st} aspect according one or more of the previous aspects, a diuretic line is connected to the source of at least one diuretic and to the patient or to the blood circuit, the diuretic pump is configured to be coupled to a pump section of the diuretic line to deliver the diuretic flow rate.

In a 42^{nd} aspect according one or more of the previous aspects, a osmotic agent line is connected to the source of at least one osmotic agent and to the patient or to the blood circuit, the osmotic agent pump is configured to be coupled to a pump section of the osmotic agent line to deliver the osmotic agent flow rate.

In a 43^{th} aspect according to aspect 41 and/or 42 when according to aspect 40, the diuretic line and/or the osmotic agent line are part of the disposable set, i.e. are grouped together and connected in the integrated disposable set.

In a 44^{th} aspect according to one or more of the previous aspects, the diuretic flow rate is between 40 mg/h and 120 mg/h (4 mg/minute).

In a 45^{th} aspect according to one or more of the previous aspects, the osmotic agent flow rate is between 10 mg/h and 150 mg/h, optionally between 25 mg/h and 100 mg/h.

In a 46^{th} aspect according to one or more of the previous aspects, the diuretic and/or the osmotic agent are administered as bolus.

In a 47^{th} aspect according to one or more of the previous aspects, the diuretic flow rate is delivered along a limited in time, in order to administer the diuretic as bolus, optionally the limited time is between 1 min and 30 min, optionally the bolus is between 40 mg and 80 mg.

In a 48^{th} aspect according to one or more of the previous aspects, the osmotic agent flow rate is delivered along a limited in time, in order to administer the diuretic as bolus, optionally the limited time is between 1 min and 30 min, optionally the bolus is between 40 mg and 80 mg.

Further characteristics of the present invention will better emerge from the detailed description that follows of some embodiments of the invention, illustrated by way of non-limiting examples in the accompanying figures.

### DESCRIPTION OF THE DRAWINGS

The description will now follow, with reference to the appended figures, provided by way of non-limiting example, in which:

FIG. 1 schematically shows an extracorporeal blood treatment apparatus according to the invention;

FIG. 2 is a different embodiment of the extracorporeal blood treatment apparatus of the invention;

FIG. 3 is a flow chart of an example of a procedure implemented by the apparatus of the invention;

FIG. 4 is a flow chart of another example of a procedure implemented by the apparatus of the invention;

FIG. 5 is a flow chart of another example of a procedure implemented by the apparatus of the invention.

### DETAILED DESCRIPTION

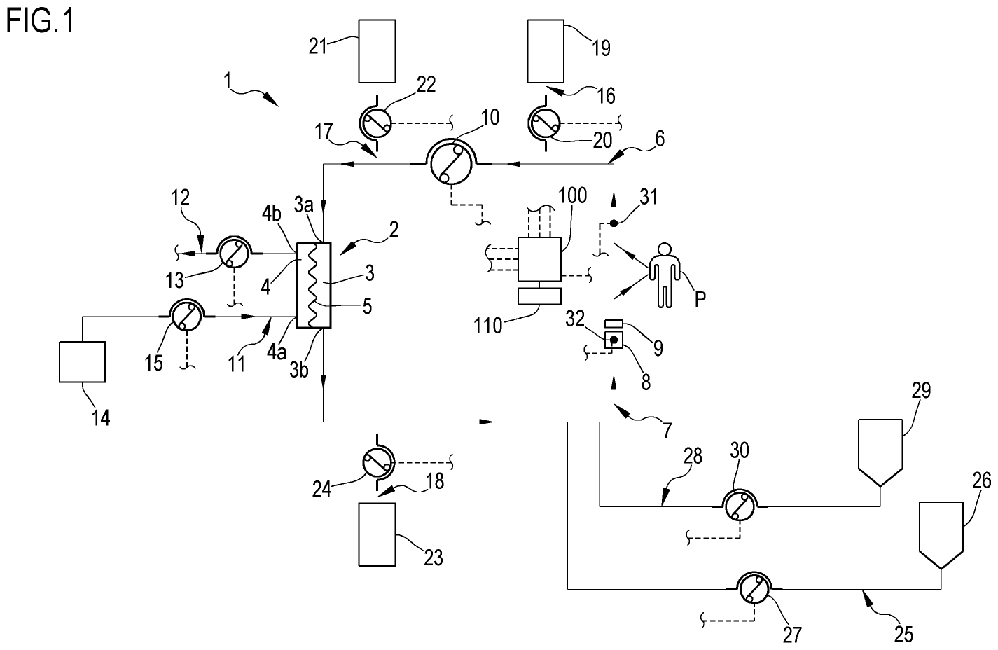

An apparatus 1 for extracorporeal blood treatment is schematically represented in FIG. 1. The apparatus 1 may be a continuous renal replacement therapy (CRRT) apparatus for intensive care treatments, for instance configured to deliver various therapies, like CCVH, CVVHDF, SCUF.

The apparatus 1 comprises a treatment or filtration unit 2 having a primary chamber 3 and a secondary chamber 4 separated by a semi-permeable membrane 5. Depending upon the treatment, the semi-permeable membrane 5 of the treatment unit 2 may be selected to have different properties and performances.

A blood circuit is coupled to the primary chamber 3 of the treatment unit 2. The blood circuit comprises a blood removal line 6 connected to an inlet 3a of the primary chamber 3, a blood return line 7 connected to an outlet 3b of the primary chamber 3.

The blood removal line 6 and blood return line 7 are configured for connection to a cardiovascular system of a patient “P”.

In use, the blood removal line 6 and the blood return line 7 are connected to a needle or to a catheter or other access device which is then placed in fluid communication with the patient “P” vascular system, such that blood may be withdrawn through the blood removal line 6, flown through the primary chamber 3 and then returned to the patient's vascular system through the blood return line 7. An air separator, such as a deaeration chamber 8, may be present on the blood return line 7. Moreover, a monitor valve 9 may be present on the blood return line 7, downstream the deaeration chamber 8.

The blood flow through the blood circuit is controlled by a blood pump 10, for instance a peristaltic blood pump, acting either on the blood removal line 6 or on the blood return line 7. The embodiment of FIG. 1 shows the blood pump 10 coupled to a pump section of the blood removal line 6.

A dialysis circuit is connected to the secondary chamber 4 of the filtration unit 2 and comprises a dialysis line 11 connected to an inlet 4a of the secondary chamber 4 and an effluent line 12 connected to an outlet 4b of the secondary chamber 4 and to a drain, not shown.

An effluent pump 13 is located on the effluent line 12 and is able to recall fluid from the second chamber 4. The dialysis line 11 is connected to a source 14, e.g. a bag or a preparation device, of fresh dialysis fluid and a dialysis pump 15 is located on the dialysis line 11 and is able to pump fluid to the second chamber 4.

The apparatus 1 further comprises an infusion circuit comprising at least one infusion line. The infusion circuit shown in the embodiment of FIG. 1 comprises a pre-blood pump line 16, a pre-infusion line 17 and a post-infusion line 18.

The pre-blood pump line 16 is connected to the blood removal line 6 upstream of the blood pump 10 and to a first source 19 of infusion fluid, e.g. a bag. A pre-blood pump 20 is located on the pre-blood pump line 16 and is able to pump fluid from the first source 19 to the blood circuit.

The pre-infusion line 17 is connected to the blood removal line 6 downstream of the blood pump 10 and upstream of the treatment unit 2 and to a second source 21 of infusion fluid, e.g. a bag. A pre-infusion pump 22 is located on the pre-infusion line 17 and is able to pump fluid from the second source 21 to the blood circuit.

The post-infusion line 18 is connected to the blood return line 7 downstream of the treatment unit 2 and to a third source 23 of infusion fluid, e.g. a bag. A post-infusion pump 24 is located on the post-infusion line 18 and is able to pump fluid from the third source 23 to the blood circuit.

The apparatus 1 may also comprise one or more auxiliary line/s, not shown, connected to the blood circuit and to a source of at least one compensation substance, such as potassium or bicarbonate, and a pump or syringe configured to deliver a flow rate of the compensation substance, such as potassium or bicarbonate.

A diuretic line 25 is connected to a source 26 of at least one diuretic and to the blood circuit. A diuretic pump 27 is configured to be coupled to a pump section of the diuretic line 25 to deliver a diuretic flow rate “Q_{d}”.

The diuretic may be or may comprise furosemide, torsemide or bumetanide, ethacrynic acid, metolazone, chlorthalidone, hydrochlorothiazide, chlorothiazide, spironolactone, eplererone.

For instance, the diuretic may be a solution of sodium chloride mixed with furosemide.

An osmotic agent line 28 is connected to a source 29 of at least one osmotic agent and to the blood circuit. An osmotic agent pump 30 is configured to be coupled to a pump section of the osmotic agent line 28 to deliver an osmotic agent flow rate “Qₒₐ”.

The osmotic agent may be or may comprise albumin, amino acid, mannitol, hypertonic saline, glucose solution.

A control unit 100 is connected and controls the blood pump 10, the effluent pump 13, the dialysis pump 15, the pre-blood pump 20, the pre-infusion pump 22 and the post-infusion pump 24 to regulate a blood flow rate “Q_{b}” in the blood circuit, a dialysis flow rate “Q_{dial}” crossing the dialysis line 11, an effluent flow rate “Q_{eff}” crossing the effluent line 12, an infusion flow rate “Q_{pbp}” crossing the pre-blood pump line 16, an infusion flow rate “Qₚᵣₑ” crossing the pre-infusion line 17, an infusion flow rate “Qₚₒₛₜ” crossing the post-infusion line 18.

Through the control of the dialysis flow rate “Q_{dial}” crossing the dialysis line 11 and/or of the effluent flow rate “Q_{eff}” crossing the effluent line 12, the control unit 100 is also configured to control/regulate a filtration flow rate “Q_{fil}” in the treatment unit 2 and/or a patient fluid removal rate “Q_{pfr}”.

The control unit 100 also controls the diuretic pump 27 and the osmotic agent pump 30 to control respectively the diuretic flow rate “Q_{d}” and the osmotic agent flow rate “Qₒₐ”.

Flow rate sensors, not shown in the attached drawings, may be placed on the blood circuit, on the dialysis line 11, on the effluent line 12, on the infusion lines 16, 17, 18, on the diuretic line 25, on the osmotic agent line 28 and connected to the control unit 100 to detect and/or control the flow rates of blood and fluids in the apparatus 1 (i.e. blood flow rate “Q_{b}”, dialysis flow rate “Q_{dial}”, effluent flow rate “Q_{eff}”, infusion flow rates “Q_{pbp}”, “Qₚᵣₑ”, “Qₚₒₛₜ”, diuretic flow rate “Q_{d}”, osmotic agent flow rate “Qₒₐ” and also filtration flow rate “Q_{fil}” and patient fluid removal rate “Q_{pfr}”).

An access pressure sensor 31 is placed on the blood removal line 6 and is connected to the control unit 100 to monitor an access pressure “P_{acc}”. A return pressure sensor 32 is placed on the blood return line 7, for instance coupled to the deaeration chamber 9, and is connected to the control unit 100 to monitor a return pressure “Pᵣₑₜ”.

Sensors are also provided to detect measured values of patient parameters, such as heart rate “HR”, blood pressure “BP”, urine output “UO”, consciousness level “CL”, body weight “BW”, creatinine “Cr”, hematocrit “Htc”, level of electrolytes. These sensors may be known and commonly used.

For instance, a heart rate sensor may be: a patch, a wristband or an armband, a chest strap, a wearable electrode based ECG. A weight scale may be used to measure the body weight “BW”. An urometer may be used for measuring urine output “UO”. An optical sensor may be used to measure hematocrit “Htc”. A digital blood pressure on the wrist, on the finger or upper arm of the patient “P”, may be used to monitor patient blood pressure “BP”. Saturation devices may also be used to retrieve information about heart rate that can indicate low blood pressure, since heart rate increases when blood pressure is getting lower (this may allow to sense a blood pressure decrease before it actually occurs). Temperature sensor to detect temperature at extremities of the patient may be used to infer blood pressure, since a low temperature means low blood pressure. For consciousness, a BIS monitor (bispectral index) may be used.

These sensors for detecting patient parameters may be connected to the control unit 100 to transmit values to the control unit 100 during the extracorporeal blood treatment (e.g. urine output “UO” and/or consciousness level “CL” and/or heart rate “HR” and/or hematocrit “Htc”). In this case, these patient parameters are measured continuously and continuously received by the control unit 100.

The sensors for detecting patient parameters may also be part of other devices which show the values such that the medical staff may enter them in the control unit 100. One or more of the patient parameters may be laboratory values (e.g. creatinine “Cr” or body weight “BW” or blood pressure “BP”). In these cases, these patient parameters are entered into the control unit 100 one or more times during the treatment or before the treatment starts.

The control unit 100 may be an electronic control unit comprising at least a CPU, a memory and input/output devices. The control unit 100 comprises or is connected to an interface 110 configured to display data and/or allow a user to input data. For instance, the interface comprises a display, e.g. a touch screen, and/or buttons or a keyboard.

The apparatus 1 may comprise a treatment machine 200 and an integrated disposable set configured to be coupled to the treatment machine.

The treatment machine comprises the cited blood pump 10, effluent pump 13, dialysis pump 15, pre-blood pump 20, pre-infusion pump 22, diuretic pump 27, osmotic agent pump 30, control unit 100 with the interface 110, flow rate sensors. The treatment machine may comprise also the access pressure sensor 31 and the return pressure sensor 32 or the access pressure sensor 31 and the return pressure sensor 32 may be part of the integrated disposable set. The treatment machine comprises also all the other elements and/or devices configured to receive and hold parts of the integrated disposable set.

The integrated disposable set comprises the treatment unit 2, the blood circuit, the effluent line 12, the dialysis line 11, the infusion lines 16, 17, 18, the diuretic line 25 and the osmotic agent line 28 which are grouped together.

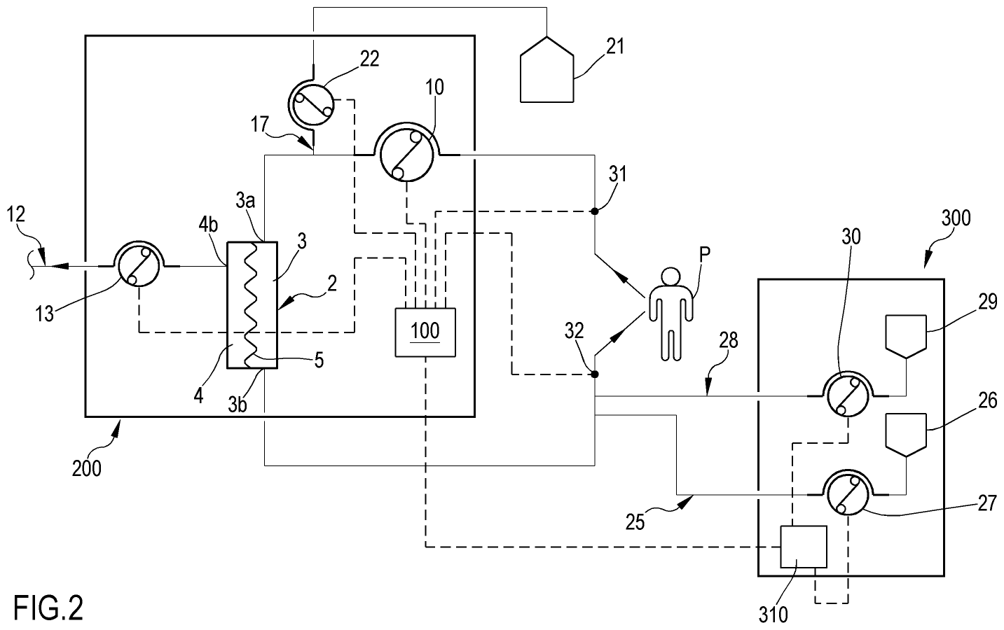

In another embodiment, shown schematically in FIG. 2, the diuretic pump 27 and the osmotic agent pump 30 are part of an independent device 300 and not of the treatment machine 200. The treatment machine 200 comprises the blood pump 10, the effluent pump 13, the pre-infusion pump 22 and the control unit 100 as detailed above regarding FIG. 1. The independent device 300 comprises the diuretic pump 27 and the osmotic agent pump 30. The diuretic pump 27 and the osmotic agent pump 30 may be connected and controlled by a control unit 310 of the independent device 300 and this control unit 310 of the independent device 300 is connected to the control unit 100 of the treatment machine 100.

In other embodiments, not shown, the diuretic line 25 and the osmotic agent line 28 may be connected directly to the vascular system of the patient “P” instead of to the blood circuit.

In a further embodiment, the treatment machine 200 may include the blood circuit (blood removal line 6 and blood return line 7), the blood pump 10, the effluent line 12, the effluent pump 13, the treatment unit 2, and the control unit 100 as detailed above regarding FIG. 2. The here described treatment machine 200 is configured for ultrafiltration (or SCUF) in the field of e.g., congestive heart failure treatments. In this specific CHF treatment, the blood removal line 6 and blood return line 7 may be connected to peripheral veins of the patient to (e.g., slowly) remove excess water. Required infusions (e.g., diuretics and/or osmotic agents) may be connected directly to the vascular system of the patient “P” instead of to the blood circuit or to the blood circuit if required/proper).

The control unit 100 is configured and/or programmed for receiving one or more input patient parameter and/or one or more input apparatus parameter and for driving, during the extracorporeal blood treatment, the diuretic pump 27 and the osmotic agent pump 30 as a function of the input patient parameter/s and/or input apparatus parameter/s, to make sure that the more optimal treatment is given to the patient “P” to achieve the more optimal fluid removal.

The control unit 100 is therefore configured for implementing a script or algorithm and a method for controlling a patient fluid removal in the apparatus for extracorporeal blood treatment.

The input patient parameters and the input apparatus parameters may be prescription values and/or values measured through the sensors and/or devices previously listed.

According to some embodiments, the control unit 100 may be configured and/or programmed for monitoring the measured values over time and driving the diuretic pump and/or the osmotic agent pump as a function of variation over time of the measured values.

According to some embodiments, the control unit is configured and/or programmed for driving the diuretic pump and/or the osmotic agent pump as a function of the prescription value of the input patient parameter/s and/or the input apparatus parameter/s, for instance as a function of a comparison between said prescription value with the measured values of the input patient parameter and/or the input apparatus parameter.

According to examples of the procedure performed by the control unit 100 or of the method, the following input parameters.

### Prescription Values

WL Total patient weight loss or fluid removal goal
T Total treatment time

### Types of Diuretic and Osmotic Agents

Diuretic: e.g. furosemide
Osmotic agent: e.g. albumin

### Measured Values

### Input Apparatus Parameters

Q_{d} diuretic flow rate
Qₒₐ osmotic agent flow rate
Q_{b} blood flow rate
Q_{pbp} infusion flow rate crossing pre-blood pump line
Qₚᵣₑ infusion flow rate crossing pre-infusion line
Qₚₒₛₜ infusion flow rate crossing post-infusion line
Q_{dial} dialysis flow rate
Q_{eff} effluent flow rate
Q_{fil} filtration flow rate
Q_{pfr} patient fluid removal rate
P_{acc} access pressure
Pᵣₑₜ return pressure
   wherein Q_{eff}=Q_{dial}+Q_{fil}=Q_{dial}+Q_{pfr}+Q_{pbp}+Qₚᵣₑ+Qₚₒₛₜ
   if Qₒₐ and Q_{d} are negligible.

### Input Patient Parameters

UO urine output (measured continuously)
CL consciousness level (measured continuously)
HR heart rate (measured continuously)
Htc hematocrit
BW Body weight (lab value)
BP Blood pressure (lab value)
Cr Creatinine (lab value)

At the start of the treatment, the following parameters are set as a function of the total patient weight loss or fluid removal goal “WT” and the total treatment time “T”.
Q_{bset} set blood flow rate
Q_{dialset} set dialysis flow rate
Q_{effset} set effluent flow rate
Q_{dset} set diuretic flow rate
P_{acc high} set high access pressure threshold
P_{acc low} set low access pressure threshold
P_{ret low} set low return pressure threshold
BPₜₕᵣ set blood pressure threshold
Htcₜₕᵣ set hematocrit threshold
Crₜₕᵣ set creatinine threshold

At the start of the treatment, the following parameters may be set equal to zero.

Qₒₐ=0
Q_{pbp}=0
Qₚᵣₑ=0
Qₚₒₛₜ=0

Therefore: Q_{fil}=Q_{eff}−Q_{dial}

The script or algorithm in the control unit 100 may be able to reach the fluid removal goal “WT” at set total treatment time “T” if no problems occur in regards of the access pressure “P_{acc}” and the blood pressure “P_{acc}”. If problems occur with the access pressure “P_{acc}” and the blood pressure “P_{acc}”, the filtration flow rate “Q_{fil}” that may be set is limited and the time to reach the goal will be longer. The script may also consider stops such as pauses in treatment, bag changes, other alarms but also recirculation etc.

The following are examples of the method implemented by the control unit 100.

If the access pressure “P_{acc}” is above the high access pressure threshold “P_{acc high}”, for instance of 250 mmHg (e.g. high access pressure indicates high blood volume), or within an access pressure range, for instance between 100 mmHg and 160 mmHg, commanding the effluent pump 13 to increase the effluent flow rate “Q_{eff}” and the filtration flow rate “Q_{fil}”.

If the access pressure “P_{acc}” is below the low access pressure threshold “P_{acc low},” (e.g. access pressure that is too low due to hypotension or either there is some hindrance in the blood removal line 6 (due for instance to folding of the catheter or kinked or twisted) or it is too close to the vascular wall or any other problem where the flow is not working properly due to the access not working), commanding the blood pump 10 to reduce the blood flow rate “Q_{b}” and/or commanding the effluent pump 13 to reduce the effluent flow rate “Q_{eff}” and the filtration flow rate “Q_{fil}” and/or commanding the osmotic agent pump 30 and/or the diuretic pump 27 to reduce or increase the diuretic flow rate “Q_{d}” and/or the osmotic agent flow rate “Qₒₐ”.

The diuretic flow rate may be between 40 mg/h and 120 mg/h (4 mg/minute) and the osmotic agent flow rate may be between 10 mg/h and 150 mg/h.

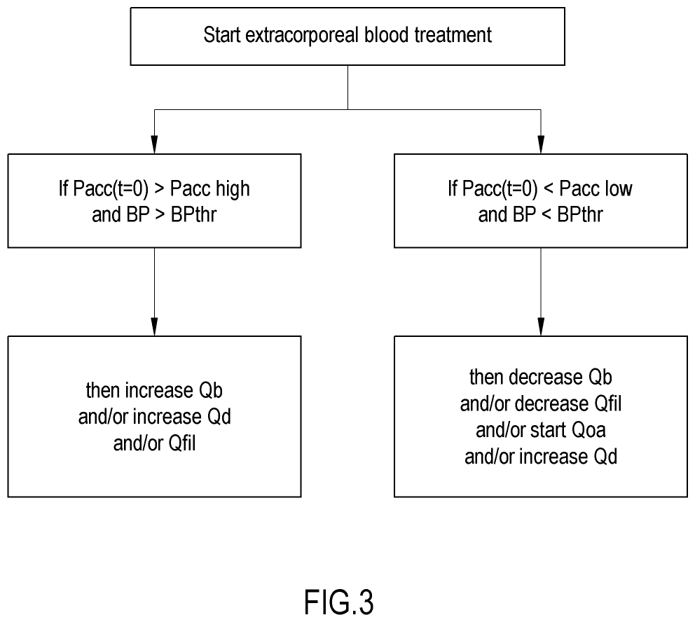

Referring to the flow chart of FIG. 3, if P_{acc}(t=0, i.e. at the start of the treatment)>P_{acc high} and BP is in an acceptable range (e.g. >BPₜₕᵣ=100 mmHg systolic), then increase Q_{b} and/or increase Q_{d} and/or Q_{fil}.

If P_{acc}(t=0)<P_{acc low}, and BP<BPₜₕᵣ=100 mmHg systolic, then decrease Q_{b} and/or decrease Q_{fil} and/or start Qₒₐ (albumin) and/or increase Q_{d} to increase blood pressure (FIG. 3).

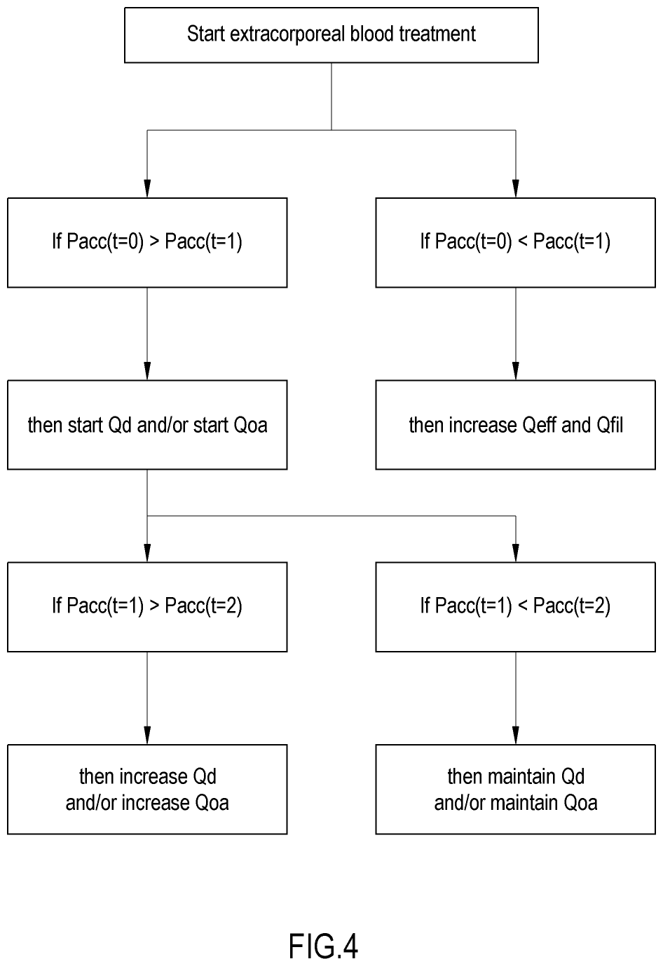

Referring to the flow chart of FIG. 4, if the access pressure “P_{acc}” decreases over time (e.g. if the access pressure at instant t=0 is greater than the access pressure at instant t=1), meaning that blood is more concentrated, i.e. there is less fluid in blood, then starting the diuretic pump 27 and/or the osmotic agent pump 30 to deliver the diuretic flow rate “Q_{d}” and/or the osmotic agent flow rate “Qₒₐ”; the osmotic agent forces fluid into the central circulation and diuretic infusion removes fluids through urine. One of the positive aspects of diuretics, such as furosemide, is that they can help remove some fluid in the lungs. If the access pressure “P_{acc}” keeps on decreasing over time (e.g. the access pressure at instant t=1 is greater than the access pressure at instant t=2), meaning that no further fluid in blood is available or not high enough concentration of diuretics is present to increase the plasma, then starting the osmotic agent pump 30 and/or the diuretic pump 27 or commanding the diuretic pump 27 to increase the diuretic flow rate “Q_{d}” and/or commanding the osmotic agent pump 30 to increase the osmotic agent flow rate “Qₒₐ”. If the access pressure “P_{acc}” starts increasing over time (e.g. if the access pressure at instant t=2 is greater than the access pressure at instant t=1), meaning that a good result has been achieved, then commanding the diuretic pump 27 to maintain the diuretic flow rate “Q_{d}” and/or commanding the osmotic agent pump 30 to maintain the osmotic agent flow rate “Qₒₐ” (FIG. 3).

If the access pressure “P_{acc}” increases over time (e.g. if the access pressure at instant t=1 is greater than the access pressure at instant t=0), taking no action or commanding the effluent pump 13 to increase the effluent flow rate “Q_{eff}” and the filtration flow rate “Q_{fil}”, in order to increase fluid removal from patient “P”. Since good conditions are present, the filtration flow rate “Q_{fil}” may be increased to remove more fluid faster (FIG. 4).

If the blood pressure “BP” is within a blood pressure range, then increase the blood flow rate “Q_{b}”, increase the diuretic flow rate “Q_{d}” and increase the effluent flow rate “Q_{eff}” and the filtration flow rate “Q_{fil}”. Since good conditions are present, the filtration flow rate may be increased to remove more fluid faster.

If the blood pressure “BP” is below a blood pressure threshold, start the osmotic agent pump 30 or commanding the osmotic agent pump 30 to increase the osmotic agent flow rate “Qₒₐ” till the blood pressure “BP” is stabilized and then starting the diuretic pump 27 or commanding the diuretic pump 27 to increase the diuretic flow rate “Q_{d}”.

If the blood pressure “BP” is low, i.e. below a blood pressure threshold, and the access pressure “P_{acc}” is outside an access pressure range, the infusion flow rate is started, e.g. of saline or glucose. Some patients may have the contradicting problem that the intravascular water concentration is low but there is an increased amount of solution in tissue (extracellular). In these conditions, addition of solution and at the same time removal of solution may be needed.

If the creatinine “Cr” is outside a creatinine range (increase in creatinine may indicate risk for damage on the kidneys) and/or the hematocrit “Htc” is outside a hematocrit range (risk for clotting), then a less aggressive treatment is performed. For instance, the blood flow rate “Q_{b}” is reduced and/or the effluent flow rate “Q_{eff}” and the filtration flow rate “Q_{fil}” are reduced while the diuretic flow rate “Q_{d}” and/or the osmotic agent flow rate “Qₒₐ” are reduced or increased, depending on the specific circumstances.

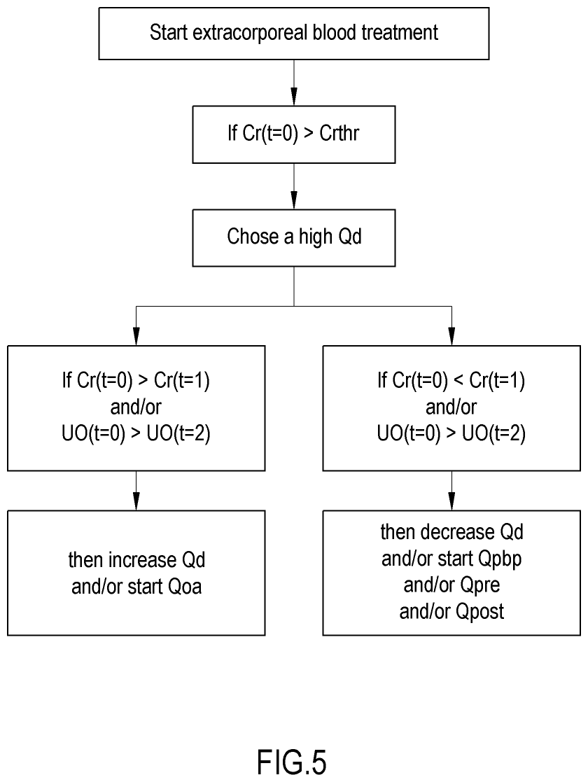

Referring to FIG. 5, if Cr(t=0) is above the creatinine threshold “Crₜₕᵣ” indicating Chronic Kidney Disease (CKD), then chose a high diuretic flow rate “Q_{d}” and then:
if Cr(t=0)>Cr(t=1) and/or UO(t=0)>UO(t=1), then increase the diuretic flow rate “Q_{d}” and/or give osmotic agent (especially if blood pressure “BP” is low);
if Cr(t=0)<Cr(t=1) and UO(t=0)>UO(t=1), then decrease the diuretic flow rate “Q_{d}” and/or give infusion fluid (especially if blood pressure “BP” is low).

If the above outlined situation of creatinine is combined with Htc (t=0)<Htc (t=1) (indicating low plasma concentration and that an extra accumulated fluid is not in the vessels but outside), then add glucose or a buffer solution or saline depending on other underlying problems the patient may have.

If Htc (t=0)<Htc (t=1), then decrease the diuretic flow rate “Q_{d}”, or, if UO (t=0)<UO (t=1), then give infusion fluid and check Htc and UO again after some time (e.g. 1 hour). If after such time the urine output “UO” is still increasing and the hematocrit “Htc” is getting better (i.e. decreasing), then continue with therapy as suggested and continue infusion of infusion fluid.

If the urine output “UO” increases, then one may increase the diuretic flow rate “Q_{d}” and adapt for the goal being reached and filtration flow rate “Q_{fil}” may be decreased, since it is always better to remove fluid through natural means.

If the access pressure “P_{acc}” is high (P_{acc}(t=0)>P_{acc high}) and the hematocrit is high (Htc>Htcₜₕᵣ) and the return pressure “Pᵣₑₜ” is low (Pᵣₑₜ<P_{ret low}), then check the access for possible clot formation.

If the access pressure “P_{acc}” is above the high access pressure threshold “P_{acc high}” or within the access pressure range and the blood pressure “BP” is greater than 100 mmHg systolic and UO (t=0)<UO (t=1) or, during the treatment, the patient shows clinical signs of fluid overload, such as shortness of breath etc., then administering a bolus (e.g. 40 mg to 80 mg) of diuretic (e.g. furosemide) and checking again blood pressure and the effect of such administration.

If heart rate “HR” starts increasing systematically and at the same time blood pressure “BP” decreases slightly, then a routine for continuous check of blood pressure is triggered or the blood flow rate “Q_{b}” and/or the diuretic flow rate “Q_{d}” is/are reduced. Then, if the blood pressure “BP” becomes stable these changes are maintained, otherwise, if the blood pressure “BP” increases, then the initial settings with higher diuretic flow rate “Q_{d}” and/or blood flow rate “Q_{b}” are recovered.

If BP<100 mmHg systolic, then give osmotic agent (e.g. albumin) and check blood pressure “BP”. If blood pressure “BP” increases, then maintain the diuretic flow rate “Q_{d}”. If blood pressure “BP” is still low, then decrease the diuretic flow rate “Q_{d}” and/or the blood flow rate “Q_{b}” and/or give infusion fluid to increase the blood pressure “BP”.

If the urine output “UO” is below a urine threshold and the blood pressure “BP” is within the blood pressure range, then increase the effluent flow rate “Q_{eff}” and the filtration flow rate “Q_{fil}” and maintain the diuretic flow rate “Q_{d}”.

If the urine output “UO” is not increasing during treatment but the blood pressure “BP” is high and the access pressure “P_{acc}” is high, then increase the blood flow rate “Q_{b}” and/or the diuretic flow rate “Q_{d}”.

If patient “P” is taking antihypertensive medication, those are to be paused during the treatment.

The script or algorithm in the control unit 100 may also check a difference between the access pressure “P_{acc}” and the return pressure “Pᵣₑₜ” during the treatment and look at the results. If the pressure difference (P_{acc}−Pᵣₑt) is getting bigger, depending on which way, it can indicate either good effect of diuretics (if fluid is added to blood from extra/intracellular liquid) or unfavorable changes (if fluid is only removed from blood and not from extra/intracellular liquid and this may cause damage to kidneys). The script or algorithm may also check the effect of osmotic agent (e.g. albumin) and the time it takes to achieve good results.

The script or algorithm in the control unit 100 may also control the effluent flow rate “Q_{eff}”, the dialysis flow rate “Q_{dial},”, the filtration flow rate “Q_{fil}” and the patient fluid removal rate “Q_{pfr}” and make sure that the changes in the diuretic flow rate “Q_{d}” and the osmotic agent flow rate “Qₒₐ” will be favorable (i.e. reduced along the treatment). If not, it will ask for e.g. creatinine “Cr” to make sure that the kidney have not worsened and/or calculate the post-filter hematocrit “Htc_{pd}” to make sure that the blood has not been concentrated too much. For instance, the post-filter hematocrit “Htc_{pd}” may be calculated through the following formula:

$$H t {c}_{p d} = {Q}_{b} \times 60 \times ( Htc / ( {Q}_{b} \times 60 + {Q}_{pbp} + {Q}_{pre} - {Q}_{fil} ) )$$

Then, the filtration flow rate “Q_{fil}” may be continued to be increased as long as hematocrit “Htc” and creatinine “Cr” have not increased too much.

The control unit 100 may also command the apparatus 1 to send an alarm signal if the access pressure is below a low alarm access pressure threshold or above a high alarm access pressure threshold.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the scope of the appended claims.

1-20. (canceled)

21. An apparatus for extracorporeal blood treatment, comprising:
a treatment unit;
a blood circuit coupled to the treatment unit and comprising a blood removal line and a blood return line connectable to a vascular system of a patient;
a blood pump coupled to a pump section of the blood circuit to deliver a blood flow rate;
an effluent line connected to the treatment unit;
an effluent pump coupled to a pump section of the effluent line to deliver an effluent flow rate;
a source of a diuretic coupled to a diuretic pump and connected to the blood circuit or connectable to the vascular system of the patient, wherein the diuretic pump is configured to deliver a diuretic flow rate;
a source of an osmotic agent coupled to an osmotic agent pump and connected to the blood circuit or connectable to the vascular system of the patient, wherein the osmotic agent pump is configured to deliver an osmotic agent flow rate;
a control unit connected to the blood pump, the diuretic pump and the osmotic agent pump and configured to receive an input patient parameter and/or an input apparatus parameter;
wherein, during an extracorporeal blood treatment, the control unit is configured to control the diuretic pump and/or the osmotic agent pump as a function of the input patient parameter and/or as a function of the input apparatus parameter.

22. The apparatus of claim 21, comprising a sensor connected to the control unit, wherein the sensor is configured to detect measured values of the input patient parameter and/or the input apparatus parameter.

23. The apparatus of claim 22, wherein the control unit is configured TO monitor said measured values over time and for controlling the diuretic pump and/or the osmotic agent pump as a function of variation over time of the measured values.

24. The apparatus of claim 21, wherein the control unit is configured to: receive a prescription value of the input patient parameter and/or the input apparatus parameter; and
control the diuretic pump and/or the osmotic agent pump as a function of the prescription value of the input patient parameter and/or the input apparatus parameter.

25. The apparatus of claim 24, wherein the control unit is configured to control the diuretic pump and/or the osmotic agent pump as a function of a comparison between the prescription value with measured values of the input patient parameter and/or the input apparatus parameter.

26. The apparatus of claim 22, wherein the sensor is configured to detect the measured values of one or more of the following input patient parameters: heart rate, blood pressure, urine output, consciousness level, body weight, creatinine, hematocrit, level of electrolytes;
wherein the control unit is configured to receive a prescription value of one or more of the following input apparatus parameter: blood flow rate, effluent flow rate, filtration flow rate;
and wherein the sensor is configured to detect the measured values of one or more of the following input apparatus parameters: blood flow rate, effluent flow rate, filtration flow rate, access pressure, return pressure.

27. The apparatus of claim 25, further comprising:
an infusion line connected to the blood circuit and to a source of an infusion fluid;
an infusion pump coupled to a pump section of the infusion line and configured to deliver an infusion flow rate;
a dialysis line connected to the treatment unit and to a source of a dialysis fluid;
a dialysis pump coupled to a pump section of the dialysis line and configured to deliver a dialysis flow rate;
wherein the control unit is configured to receive the prescription value of the one or more of the following input apparatus parameter: infusion flow rate and dialysis flow rate.

28. The apparatus of claim 27, wherein the sensor is configured to detect the measured values of one or more of the following input apparatus parameters: infusion flow rate and dialysis flow rate.

29. The apparatus of claim 26, wherein the control unit is configured to perform the following procedure:
when, at the start of the extracorporeal blood treatment, the access pressure is above a high access pressure threshold and the blood pressure is above a blood pressure threshold or within an access pressure range, the control unit is configured to operate the blood pump to increase the blood flow rate and/or operate the effluent pump to increase the effluent flow rate and the filtration flow rate and/or operate the diuretic pump to start or increase the diuretic flow rate;
when, at the start of the extracorporeal blood treatment, the access pressure is below a low access pressure threshold and the blood pressure is below a blood pressure threshold, the control unit is configured to operate the blood pump to decrease the blood flow rate and/or operate the effluent pump to decrease the effluent flow rate and the filtration flow rate and/or operate the osmotic agent pump to start or increase the osmotic agent and/or operate the diuretic pump to start or increase the diuretic flow rate.

30. The apparatus of claim 29, wherein the control unit is configured to perform the following procedure:
when the access pressure decreases over time, the control unit is configured to operate the diuretic pump and/or the osmotic agent pump to deliver the diuretic flow rate and/or the osmotic agent flow rate; or
when the access pressure increases over time, the control unit is configured to take no action or operate the effluent pump to increase the effluent flow rate and the filtration flow rate.

31. The apparatus of claim 26, wherein, when, at the start of the extracorporeal blood treatment, the creatinine is above a selected creatinine threshold indicating chronic kidney disease, the control unit is configured to operate the diuretic pump to increase the diuretic flow rate.

32. The apparatus of claim 21, wherein the control unit is configured to perform the following procedure:
when the creatinine starts decreasing over time and/or the urine output starts decreasing over time, the control unit is configured to operate the diuretic pump to increase the diuretic flow rate and/or operate the osmotic pump to start;
when the creatinine starts increasing over time and/or the urine output starts decreasing over time, the control unit is configured to operate the diuretic pump to decrease the diuretic flow rate.

33. The apparatus of claim 22, further comprising:
an infusion line connected to the blood circuit and to a source of an infusion fluid;
an infusion pump coupled to a pump section of the infusion line;
wherein, when the creatinine starts increasing over time and/or the urine output starts decreasing over time, the control unit is configured to operate the infusion pump to deliver the infusion fluid through the infusion line at an infusion flow rate.

34. The apparatus of claim 26, wherein the control unit is configured to perform the following procedure:
when hematocrit increases over time, the control unit is configured to operate the diuretic pump to decrease the diuretic flow rate; or
when the urine output increases over time, the control unit is configured to operate the infusion pump to start or to increase the infusion flow rate, and, if the urine output still increases over time and hematocrit starts decreasing, the control unit is configured to operate the infusion pump to maintain the infusion flow rate.

35. The apparatus of claim 21, wherein the control unit is configured to receive data of the diuretic and/or data of the osmotic agent.

36. The apparatus of claim 21, wherein the osmotic agent comprises albumin or amino acids or mannitol or hypertonic saline or glucose solution; and wherein the diuretic comprises furosemide or torsemide or bumetanide or ethacrynic acid or metolazone or chlorthalidone or hydrochlorothiazide or chlorothiazide or spironolactone or eplererone.

37. The apparatus of claim 21, wherein the apparatus comprises a treatment machine including the control unit and the blood pump, wherein the treatment machine further comprises the osmotic agent pump and/or the diuretic pump.

38. The apparatus of claim 21, wherein the apparatus comprises a treatment machine including the control unit and the blood pump, wherein the osmotic agent pump and/or the diuretic pump is/are external to the treatment machine.

39. The apparatus of claim 21, wherein a diuretic line is connected to the source of a diuretic and either to the patient or to the blood circuit, the diuretic pump being coupled to a pump section of the diuretic line to deliver the diuretic flow rate;
wherein an osmotic agent line is connected to the source of an osmotic agent and either to the patient or to the blood circuit, the osmotic agent pump being coupled to a pump section of the osmotic agent line to deliver the osmotic agent flow rate;
wherein the apparatus comprises an integrated disposable set, wherein the treatment unit, the blood circuit, and the effluent line are grouped together and connected in the integrated disposable set, wherein one or more of the diuretic line and the osmotic agent line is connected to the integrated disposable set.

40. The apparatus of claim 21, further comprising:
an auxiliary line connected to the blood circuit and to a source of a compensation substance comprising potassium and/or bicarbonate;
a pump or syringe coupled to a pump section of the auxiliary line and configured to deliver a flow rate of the compensation substance;
wherein the input apparatus parameter further comprises the flow rate of the compensation substance.

41. The apparatus of claim 21, wherein the control unit is configured to pause delivery of antihypertensive medication during the extracorporeal blood treatment.

42. The apparatus of claim 30, wherein, when the access pressure decreases over time, the control unit is configured to perform the following procedure:
when the access pressure keeps on decreasing over time, the control unit is configured to:
start operating the osmotic agent pump or operate the osmotic agent pump to increase the osmotic agent flow rate; and/or
start operating the diuretic pump or operate the diuretic pump to increase the diuretic flow rate;
when the access pressure starts increasing over time, the control unit is configured to:
operate the diuretic pump to maintain the diuretic flow rate; and/or
operate the osmotic agent pump to maintain the osmotic agent flow rate.

43. The apparatus of claim 26, wherein:
when the blood pressure is within a selected blood pressure range, the control unit is configured to: operate the blood pump to increase the blood flow rate, operate the diuretic pump to increase the diuretic flow rate, and operate the effluent pump to increase the effluent flow rate and the filtration flow rate;
when the blood pressure is below a selected blood pressure threshold, the control unit is configured to: start operating the osmotic agent pump or operate the osmotic agent pump to increase the osmotic agent flow rate until the blood pressure is stabilized, and start operating the diuretic pump or operate the diuretic pump to increase the diuretic flow rate.

44. The apparatus of claim 21, wherein the apparatus is a continuous renal replacement therapy system for intensive care treatments.

45. An apparatus for extracorporeal blood treatment, comprising:
a treatment unit;
a blood circuit coupled to the treatment unit and comprising a blood removal line and a blood return line connectable to a vascular system of a patient;
a blood pump coupled to a pump section of the blood circuit to deliver a blood flow rate;
an effluent line connected to the treatment unit;
an effluent pump coupled to a pump section of the effluent line to deliver an effluent flow rate;
a source of a diuretic coupled to a diuretic pump and connected to the blood circuit or connectable to the vascular system of the patient, wherein the diuretic pump is configured to deliver a diuretic flow rate;
a source of an osmotic agent coupled to an osmotic agent pump and connected to the blood circuit or connectable to the vascular system of the patient, wherein the osmotic agent pump is configured to deliver an osmotic agent flow rate;
a sensor;
a control unit operably connected to the sensor, the blood pump, the diuretic pump and the osmotic agent pump, the control unit configured to receive an input patient parameter and an input apparatus parameter, the sensor configured to detect measured values of the input patient parameter and/or the input apparatus parameter;
wherein the input patient parameters detected by the sensor comprise one or more of a heart rate, a blood pressure, a urine output, a consciousness level, a body weight, a creatinine value, a hematocrit value, and a level of electrolytes;
wherein the control unit is configured to receive a prescription value of one or more of the following input apparatus parameters: blood flow rate, effluent flow rate, and filtration flow rate; and the sensor is configured to detect the measured values of one or more of the following input apparatus parameters: blood flow rate, effluent flow rate, filtration flow rate, access pressure, and return pressure;
wherein the control unit is configured to check the access pressure and the blood pressure at the start of the extracorporeal blood treatment and perform the following procedure:
when, at the start of the extracorporeal blood treatment, the access pressure is above a selected high access pressure threshold and the blood pressure is above a selected blood pressure threshold or within a selected access pressure range, the control unit is configured to operate one or more of:
the blood pump to increase the blood flow rate,
the effluent pump to increase the effluent flow rate,
the diuretic pump to start or increase the diuretic flow rate;
when, at the start of the extracorporeal blood treatment, the access pressure is below a selected low access pressure threshold and the blood pressure is below a selected blood pressure threshold, the control unit is configured to operate one or more of:
the blood pump to decrease the blood flow rate,
the effluent pump to decrease the effluent flow rate,
the osmotic agent pump to start or increase delivery of the osmotic agent,
the diuretic pump to start or increase the diuretic flow rate.
